(19) United States
(12) Patent Application Publication
(10) Pub. No.: US 2019/0194862 A1
(43) Pub. Date: Jun. 27, 2019

(54) Method for having cleaner dried laundry

(71) Applicant: Vera Ann Flory, West Alexandria, OH (US)

(72) Inventor: Vera Ann Flory, West Alexandria, OH (US)

(73) Assignee: VA DESIGNS, Ltd, West Alexandria, OH (US)

(21) Appl. No.: 16/350,725

(22) Filed: Dec. 21, 2018

Related U.S. Application Data
(60) Provisional application No. 62/708,859, filed on Dec. 26, 2017.

(51) Int. Cl. D06F 58/20; A61L 2/00

(52) U.S. Cl. CPC D06F 58/203; D06F 58/02; A61L 2/00

(57) ABSTRACT

This method for cleaner, fluffier, better smelling laundry is a preferred method over prior art because it is ecology safer, saves money and energy resources while removing toxins and generally making life more pleasant. The object of this invention is to have cleaner, fluffier dried laundry by detoxifying, deodorizing and disinfecting. This makes the laundry genuinely clean as well as improving the quality of the environment and can be used as a stand alone cleaner if the clothes are only nose telling dirty.

### BACKGROUND OF THE INVENTION

a—Field of Invention

This invention generally relates to a method for having fluffier, cleaner, better smelling, dried laundry.

b—Background

People use smelly, fabric softener in their dryer. Even their washed laundry can smell of chemicals used to clean the clothes. And there are some other smells that don't seem to disappear for a long time.

### BRIEF SUMMARY OF THE INVENTION

The object of this invention is to have fluffier, cleaner, better smelling dried laundry that is detoxified, deodorized and disinfected, without using harmful, smelly chemicals. Another object of this invention is to save money, energy resources, the quality of the environment and the launderer's health, while removing toxins and generally making life more pleasant. Another way this invention saves energy resources is that some of the laundry may not even have to be washed. Especially the laundry that just smells dirty. It can come out of the dryer detoxified, deodorized and disinfected.

### BRIEF DESCRIPTION OF THE VIEWS OF THE DRAWING

FIG. 1 shows a laundry dyer machine. While there are many brands, shapes, and sizes, the door and the control panel is not always located at the same place. This drawing shows a dryer with the control panel near the back just as an illustration.

FIG. 2 #1 shows the shroud of a dryer.

FIG. 2 #2 shows the dryer drum or tumbler. While there are many brands, shapes, and sizes, the drier drum can be at a different angle. This drawing shown is just for an illustration of how there is space between a circular shape and a square shape. The apparatus could be anywhere if so desired. Said apparatus could even be outside the dryer with a means to get air into the dryer.

FIG. 2 #3 shows the opening for loading and unloading the dryer.

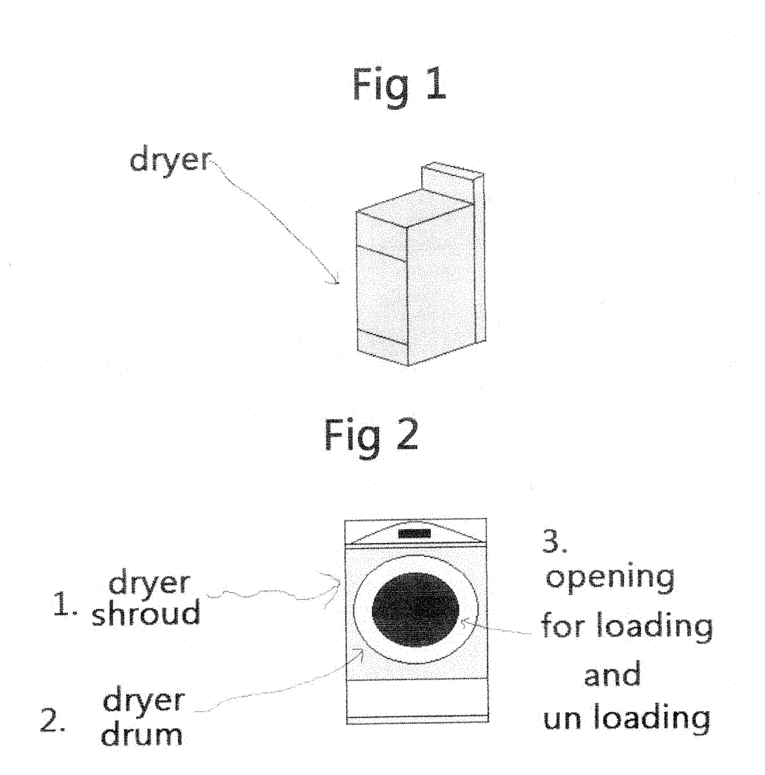

### DETAILED DESCRIPTION OF THE INVENTION

This method for cleaner, fluffier laundry is a preferred method over prior art because it is ecology safer, whereby destroying mold, mildew, tobacco, body odors, fragrances and other offensive smells of which most are toxic. The quality of the environment can be helped by using this invention in removing toxins, bacteria and bad odors and generally making life more pleasant.

This method to have cleaner, fluffier, better smelling dried laundry uses an apparatus with a dryer, called Lightning Clean Technology™. Said apparatus detoxifies, deodorizes and disinfects. It works kind of like after a thunder storm in that it leaves a clean spring-rain smell.

The apparatus works with the appliance control panel through wiring, radio waves, or any wireless methods. The apparatus could even be outside the dryer with a means to get air into the dryer, if so desired. This air then goes through the dryer and out through the vent to outdoors or some place where people are not

What is claimed is:

1. A method for getting clothes cleaner by using an apparatus called Lightning Clean Technology™ connecting to a drier.
